(19) United States
(12) Patent Application Publication
(10) Pub. No.: US 2021/0311015 A1
(43) Pub. Date: Oct. 7, 2021

(54) DETECTING DEVICE

(71) Applicant: SPEED TECH CORPORATION, Taoyuan City (TW)

(72) Inventors: Chih-Cheng CHEN, Taoyuan City (TW); Kuo-Hua Huang, Taoyuan City (TW); Yung-Lung Hsu, Taoyuan City (TW)

(73) Assignee: SPEED TECH CORPORATION, Taoyuan City (TW)

(21) Appl. No.: 17/036,477

(22) Filed: Sep. 29, 2020

(30) Foreign Application Priority Data
Apr. 7, 2020 (TW) 109111667

(51) Int. Cl. G01N 33/487; H01R 12/71

(52) U.S. Cl. CPC G01N 33/4875; H01R 2201/12; H01R 12/71

(57) ABSTRACT

The present disclosure provides a detecting device, comprising a test strip connector, a sliding block, an elastomer, a housing, and an elastic piece. The sliding block is slidably disposed on the test strip connector. The sliding block comprises an elastic piece connecting part. The elastomer is disposed between the sliding block and the test strip connector. The housing is disposed on the test strip connector and comprises a first connecting part. The elastic piece connecting part is exposed from the housing. The elastic piece comprises a second connecting part, a connecting end part, and a pressing end part. The connecting end part and the pressing end part are disposed at two sides of the second connecting part. The second connecting part is rotatably connected to the first connecting part. The connecting end part is movably connected to the elastic piece connecting part.

### CROSS REFERENCE TO RELATED APPLICATION

This application claims the priority benefit of Taiwanese Patent Application Serial Number TW109111667, filed on Apr. 7, 2020, the full disclosure of which is incorporated herein by reference.

### BACKGROUND

### Technical Field

The present disclosure relates to the technical field of test strip detection, particularly to a detecting device.

### Related Art

The steps of conventional detecting device to detect samples on a test strip is firstly to take samples from the human body (such as blood, urine, or saliva, etc.) with a test strip, then the test strip is inserted into a detecting device which would detect and analyze the sample. After the detection is completed, the samples can be withdrawn from the detecting device. However, hands are likely to touch the samples on test strips to possibly cause infection when withdrawing test strips.

### SUMMARY

The embodiments of the present disclosure provide a detecting device to solve the problem of touching samples on test strips causing infection as the test strips are withdrawn from detecting device by hand.

The embodiments of the present disclosure provide a detecting device, comprising a test strip connector, a sliding block, an elastomer, a housing and an elastic piece. The sliding block is slidably disposed on the test strip connector comprising an elastic piece connecting part. The elastomer is disposed between the sliding block and the test strip connector. The extending direction of the elastomer is parallel to the sliding direction of the sliding block. The housing is disposed on the test strip connector and comprises a first connecting part and a first opening. The elastic piece connecting part of the sliding block is exposed from the first opening. The elastic piece comprises a second connecting part, a connecting end part, and a pressing end part. The connecting end part and the pressing end part are disposed on two sides of the second connecting part rotatably connected to the first connecting part. The connecting end part is movably connected to the elastic piece connecting part. Wherein an end part of a test strip is inserted into the test strip connector. The connecting end part is separated from the elastic piece connecting part after the pressing end part is pressed. The elastomer pushes the sliding block which drives an end part of the test strip out of the test strip connector.

In the embodiments of the present disclosure, by disposing a sliding block and an elastomer on a test strip connector and by controlling the position of the sliding block through an elastic piece, a detecting device can detect the sample on the test strip when the elastic piece is connected to the sliding block. When the elastic piece is detached from the sliding block, the sliding block can be pushed by the elastomer to allow automatically returning of the test strip.

It should be understood, however, that this summary may not contain all aspects and embodiments of the present disclosure, that this summary is not meant to be limiting or restrictive in any manner, and that the disclosure as disclosed herein will be understood by one of ordinary skill in the art to encompass obvious improvements and modifications thereto.

### BRIEF DESCRIPTION OF THE DRAWINGS

The features of the exemplary embodiments believed to be novel and the elements and/or the steps characteristic of the exemplary embodiments are set forth with particularity in the appended claims. The Figures are for illustration purposes only and are not drawn to scale. The exemplary embodiments, both as to organization and method of operation, may best be understood by reference to the detailed description which follows taken in conjunction with the accompanying drawings in which:

FIG. 1 is a perspective view of a detecting device of the first embodiment of the present disclosure;

FIG. 2 is an exploded view of the detecting device of the first embodiment of the present disclosure;

FIG. 3 is a cross-sectional view along line A-A′ of FIG. 1;

FIG. 4 is an enlarged view of area A of FIG. 3;

FIG. 5 is an enlarged view of area B of FIG. 4;

FIG. 6 is a combined perspective view of an elastic piece, a base plate, a housing and a test strip connector of the first embodiment of the present disclosure;

FIG. 7 is a cross-sectional view of a detecting device of the second embodiment of the present disclosure;

FIG. 8 is a combined perspective view of an elastic piece, a base plate, a housing and a test strip connector of the third embodiment of the present disclosure;

FIG. 9 is a combined perspective view of an elastic piece, a base plate, a housing and a test strip connector of the fourth embodiment of the present disclosure;

FIG. 10 is a partially exploded view of an elastic piece, a base plate, a housing and a test strip connector of the fourth embodiment of the present disclosure;

FIG. 11 is a combined perspective view of an elastic piece, a base plate, a housing and a test strip connector of the fifth embodiment of the present disclosure; and

FIG. 12 is a combined perspective view of an elastic piece, a base plate, a housing and a test strip connector of the sixth embodiment of the present disclosure.

### DETAILED DESCRIPTION OF THE EMBODIMENTS

The present disclosure will now be described more fully hereinafter with reference to the accompanying drawings, in which exemplary embodiments of the disclosure are shown. This present disclosure may, however, be embodied in many different forms and should not be construed as limited to the embodiments set forth herein. Rather, these embodiments are provided so that this present disclosure will be thorough and complete, and will fully convey the scope of the present disclosure to those skilled in the art.

Certain terms are used throughout the description and following claims to refer to particular components. As one skilled in the art will appreciate, manufacturers may refer to a component by different names. This document does not intend to distinguish between components that differ in name but function. In the following description and in the claims, the terms “include/including” and “comprise/comprising” are used in an open-ended fashion, and thus should be interpreted as “including but not limited to”. “Substantial/substantially” means, within an acceptable error range, the person skilled in the art may solve the technical problem in a certain error range to achieve the basic technical effect.

The following description is of the best-contemplated mode of carrying out the disclosure. This description is made for the purpose of illustration of the general principles of the disclosure and should not be taken in a limiting sense. The scope of the disclosure is best determined by reference to the appended claims.

Moreover, the terms “include”, “contain”, and any variation thereof are intended to cover a non-exclusive inclusion. Therefore, a process, method, object, or device that includes a series of elements not only includes these elements, but also includes other elements not specified expressly, or may include inherent elements of the process, method, object, or device. If no more limitations are made, an element limited by “include a/an . . . ” does not exclude other same elements existing in the process, the method, the article, or the device which includes the element.

In the following embodiment, the same reference numerals are used to refer to the same or similar elements throughout the disclosure.

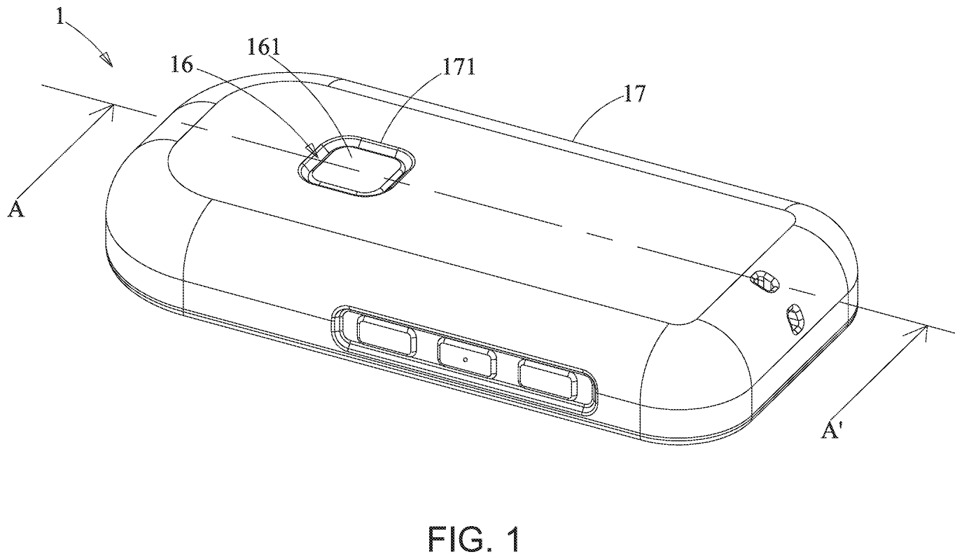

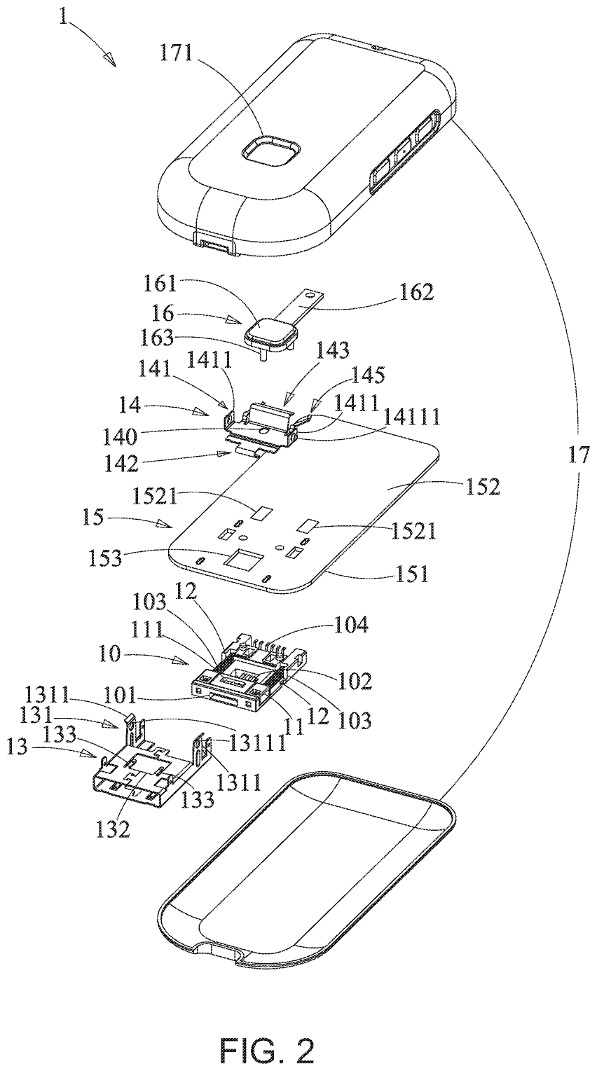

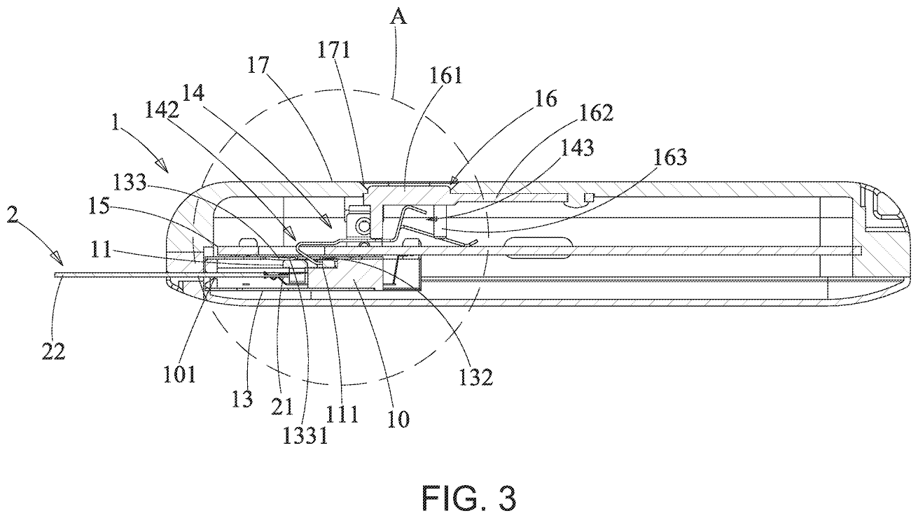

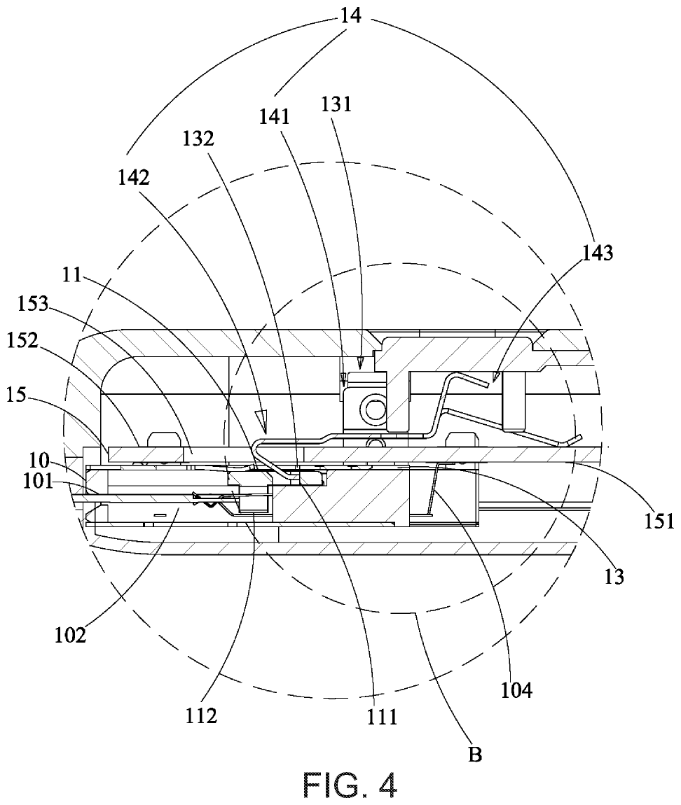

FIG. 1 and FIG. 2 are perspective view and exploded view of a detecting device of the first embodiment of the present disclosure. FIG. 3 is a cross-sectional view along line A-A′ of FIG. 1. FIG. 4 is an enlarged view of area A of FIG. 3. As shown in the figure, the detecting device 1 of this embodiment comprises a test strip connector 10, a sliding block 11, an elastomer 12, a housing 13, and an elastic piece 14. The test strip connector 10 comprises a test strip insertion hole 101 and a test strip accommodating space 102. The test strip insertion hole 101 is in communicating with the test strip accommodating space 102. The sliding block 11 is slidably disposed on the test strip connector 10. The initial position of the sliding block 11 (the position where the test strip is not inserted, as shown in FIG. 2) is disposed on one side of the test strip accommodating space 102 close to the test strip insertion hole 101. The sliding block 11 comprises an elastic piece connecting part 111 and a pushing part 112. The pushing part 112 is disposed in the test strip accommodating space 102. The elastomer 12 is disposed between the sliding block 11 and the test strip connector 10. The extending direction of the elastomer 12 is parallel to the sliding direction of the sliding block 11. The housing 13 is disposed on the test strip connector 10 and comprises a first connecting part 131 and a first opening 132. The elastic piece connecting part 111 of the sliding block 11 is exposed from the first opening 132. The elastic piece 14 comprises a second connecting part 141, a connecting end part 142, and a pressing end part 143. The connecting end part 142 and the pressing end part 143 are disposed on two sides of the second connecting part 141 rotatably connected to the first connecting part 131. The connecting end part 142 is movably connected to the elastic piece connecting part 111. Wherein an end part of a test strip is inserted into the test strip connector. The connecting end part 142 is separated from the elastic piece connecting part 111 when the sliding block 11 is in the initial position.

The test strip 2 can be inserted into the strip connector 10 for testing. The test strip 2 comprises a test strip connecting end part 21 and a sampling end part 22. The test strip connecting end part 21 of the test strip 2 is inserted into the test strip accommodating space 102 through the test strip insertion hole 101 of the test strip connector 10. The test strip connecting end part 21 pushes the pushing part 112 of the sliding block 11 to move away from the test strip insertion hole 101. The sliding block 11 compresses the elastomer 12. Finally, the sliding block 11 is disposed on one side of the test strip accommodating space 102 away from the test strip insertion hole 101 and allows the elastic piece connecting part 111 of the sliding block 11 to connect with the connecting end part 142 of the elastic piece 14 (as shown in FIG. 4). When the pressing end part 143 is pressed, the pressing end part 143 moves in a direction toward the housing 13. The second connecting part 141 of the elastic piece 14 rotates relative to the first connecting part 131 of the housing 13. The connecting end part 142 of the elastic piece 14 is separated from the elastic piece connecting part 111 of the sliding block 11 and moves in a direction away from the housing 13. The compressed elastomer 12 is then released. The elastic force of the elastomer 12 pushes the sliding block 11 toward the test strip insertion hole 101. The sliding block 11 also pushes the test strip connecting end 21 of the test strip 2 toward the outside of the test strip connector 10. So, the detecting device 1 could perform the function of automatically returning of the test strip.

In this embodiment, the test strip connector 10 further comprises two sliding grooves 103 respectively disposed on two sides of the test strip accommodating space 102. The extending direction of the sliding groove 103 is perpendicular to the extending direction of the test strip insertion hole 101. The two sides of the sliding block 11 are respectively disposed in two sliding groove 103. The number of the elastomer 12 is two, and each elastomer 12 is disposed in the corresponding sliding groove 103. Two ends of the elastomer 12 are respectively in contact with the surfaces of the sliding block 11 and the sliding groove 103 away from the test strip insertion hole 101. The elastomer 12 in this embodiment is a spring. The elastomer 12 could also be an elastic pad, an elastic piece, or other elastomers.

The detecting device of this embodiment further comprises a base plate 15 comprising a first surface 151, a second surface 152, and a second opening 153. The first surface 151 is opposite to the second surface 152. The second opening 153 passes through the first surface 151 and the second surface 152. The test strip connector 10 is disposed on the first surface 151. The first connecting part 131 of the housing 13 passes through the base plate 15. The first connecting part 131 protrudes from the second surface 152. The second opening 153 corresponds to the first opening 132. The elastic piece connecting part 111 is exposed from the second opening 153. The second connecting part 141 of the elastic piece 14 is rotatably connected to the first connecting part 131 of the housing 13. The elastic piece 14 is disposed on the second surface 152. The connecting end part 142 of the elastic piece 14 is movably connected to the elastic piece connecting part 111.

The base plate 15 of this embodiment is a circuit board. The test strip connector 10 comprises a plurality of terminals 104. One end of the terminals 104 is electrically connected to the base plate 15. The other end of the terminals 104 is disposed in the test strip accommodating space 102. When the test strip 2 is inserted into the test strip accommodating space 102 of the test strip connector 10, the test strip connecting end part 21 of the test strip 2 is connected to the terminals 104 disposed in the test strip accommodating space 102. In this way, the detection circuit of the base plate 15 can detect the samples at the sampling end part 22.

The detecting device of this embodiment further comprises a button 16 and an outer housing 17. The button 16 is disposed on one side of the elastic piece 14 away from the connecting end part 142 and is disposed on one side of the pressing end part 143. In this embodiment, the button 16 is disposed on the pressing end part 143 and is opposite to the base plate 15. The pressing end part 143 can be pressed by pressing the button 16. The outer housing 17 accommodates the test strip connector 10, the sliding block 11, the elastomer 12, the housing 13, the elastic piece 14, and the base plate 15. The outer housing 17 comprises a button opening 171. The button 16 is disposed in the button opening 171. In this embodiment, the button 16 comprises a button body 161 and a securing piece 162 connected to one side of the button body 161. The securing piece 162 extends in a direction away from the button body 161. The button body 161 is disposed in the button opening 1741. The securing piece 162 is secured to an inner surface of the outer housing 17. In one embodiment, the button 16 further comprises at least one limiting cylinder 163 disposed on a surface of the button body 161 facing the base plate 15. When the button 16 is pressed, the button body 161 moves into the outer housing 17. One end of the limiting cylinder 163 away from the button body 161 contacts the second surface 152 of the base plate 15. Then, the button body 161 stops moving into the outer housing 17. In other words, the limiting cylinder 163 could limit the moving stroke of the button body 161. When the limiting cylinder 163 is desired to pass through the elastic piece 14, it can pass through a retaining hole 140 provided on the elastic piece 14. Thus, the operation of the elastic piece 14 would not be affected.

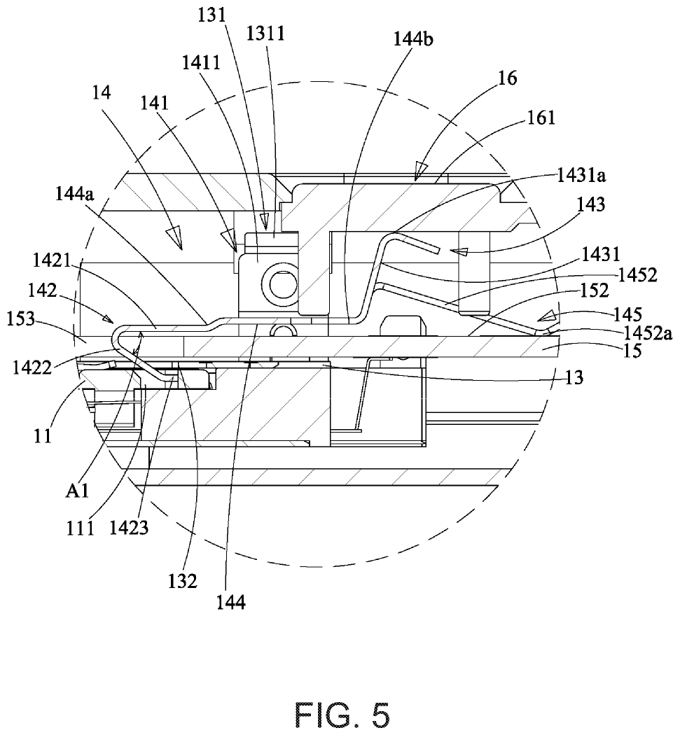

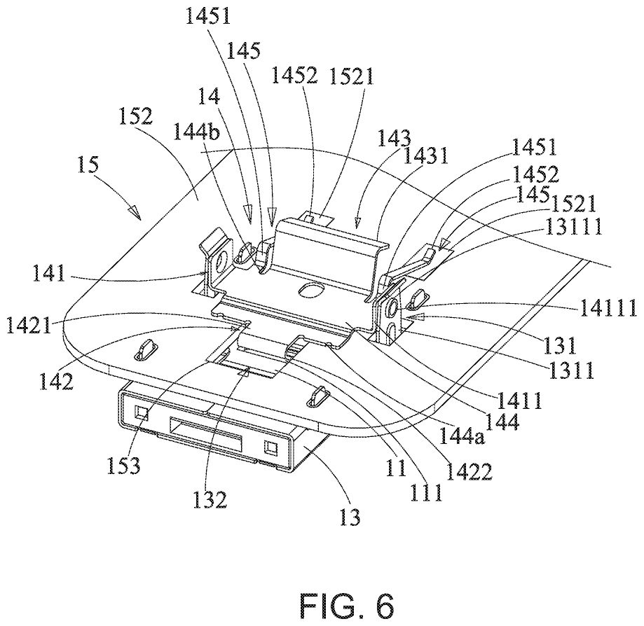

FIG. 5 is an enlarged view of area B of FIG. 4. FIG. 6 is a combined perspective view of an elastic piece, a base plate, a housing, and a test strip connector of the first embodiment of the present disclosure. As shown in the figure, the elastic piece 14 of this embodiment comprises an elastic piece body 144 comprising a first side 144a and a second side 144b opposite to the first side 144a. The second connecting part 141 is disposed on the elastic piece body 144, i.e., on one side between the first side 144a and the second side 144b. The connecting end part 142 is connected to the first side 144a and is disposed on one side of the elastic piece body 144. The pressing end 143 is connected to the second side 144b and is disposed on the other side of the elastic piece body 144. The first connecting part 131 of this embodiment comprises two first connecting arms 1311 oppositely disposed. Each of the first connecting arms 1311 comprises a connecting hole 13111. The second connecting part 141 comprises two second connecting arms 1411 oppositely disposed. Each of the second connecting arms 1411 comprises a connecting shaft 14111. The connecting shaft 14111 of each of the second connecting arms 1411 is disposed in the connecting hole 13111 of the corresponding first connecting arm 1311. In this way, the first connecting part 131 can be rotatably connected to the second connecting part 141, and the second connecting part 141 could rotate relative to the first connecting part 131. In this embodiment, the second connecting part 141 of the elastic piece 14 is assembled to the first connecting part 131 of the housing 13 along a direction perpendicular to the second surface 152 of the base plate 15.

The connecting end part 142 of this embodiment comprises a first connecting piece 1421 and a second connecting piece 1422. The first connecting piece 1421 is connected to a first side 144a of the elastic piece body 144. The second connecting piece 1422 is connected to one side of the first connecting piece 1421 away from the elastic piece body 144 and extends in a direction of the test strip accommodating space 102. One side of the second connecting piece 1422 away from the first connecting piece 1421 is movably connected to the elastic piece connecting part 111. One side of the second connecting piece 1422 of this embodiment away from the first connecting piece 1421 passes through the second opening 153 of the base plate 15 and the first opening 132 of the housing 13 and abuts against a sidewall of the elastic piece connecting part 111. In this embodiment, an angle A1 is defined between the second connecting piece 1422 and the first connecting piece 1421, in which the angle A1 is greater than 45 degrees and less than 90 degrees. Meanwhile, the first connecting piece 1421 and the second connecting piece 1422 are integrally formed to one piece. The second connecting piece 1422 could firmly abut against a sidewall of the elastic piece connecting part 111 of the sliding block 11. That is, the force exerted by the second connecting piece 1422 on the sliding block 11 is equal to or greater than the elastic force of the elastomer 12.

In one embodiment, the connecting end 142 further comprises a third connecting piece 1423 connected to the side of the second connecting piece 1422 away from the first connecting piece 1421. When the connecting end part 142 is connected to the elastic piece connecting part 111 of the sliding block 11, the first connecting piece 1421 is substantially parallel to the second surface 152 of the base plate 15, and the third connecting piece 1423 is parallel to the first connecting piece 1421. So, the third connecting piece 1423 abuts against a sidewall of the elastic piece connecting part 111. In this embodiment, the third connecting piece 1423 is perpendicular to a sidewall of the elastic piece connecting part 111. The forcing direction of the third connecting piece 1423 is perpendicular to a sidewall of the elastic piece connecting part 111. Before pressing the pressing end part 143, the connecting end part 142 firmly presses against a sidewall of the elastic piece connecting part 111. So, the sliding block 11 is disposed on one side of the test strip accommodating space 102 away from the test strip insertion hole 101.

The pressing end part 143 of this embodiment comprises a pressing elastic piece 1431 connected to the second side 144b of the elastic piece body 144. The pressing elastic piece 1431 extend toward the button 16. One side of the pressing elastic piece 1431 away from the elastic piece body 144 is disposed on one side of the button 16. In one embodiment, one side of the pressing elastic piece 1431 away from the elastic piece body 144 comprises a first bending part 1431a disposed on one side of the button 16. That is, the first bending part 1431a is closer than any other parts to the button 16. When the button 16 is pressed, the button body 161 would first contact the first bending part 1431a. By pressing the first bending part 1431a, the pressing elastic piece 1431 would move in a direction closing to the housing 13. The surface of the first bending part 1431a facing the button 16 is arc-shaped, which increases the contact area between the button 16 and the first bending part 1431a and avoid wearing of the pressing elastic piece 1431 by contacting with the button 16.

The elastic piece 14 of this embodiment further comprises two supporting end parts 145. The two supporting end parts 145 are on the same side as the pressing end part 143, which means the supporting end part 145 is connected to the second side 144b of the elastic piece body 144. The two supporting end parts 145 are disposed on two sides of the pressing end part 143 and is connected to the pressing end part 143 through the elastic piece body 144. That is, the second supporting end part 145 is not directly connected to the pressing end part 143, and the second supporting end part 145 abuts against the second surface 152 of the base plate 15. When the pressing end part 143 is pressed by the button 16, the pressing end part 143 moves in a direction toward the base plate 15. One end of the two supporting end parts 145 abutting against the base plate 15 moves on the second surface 152 and move in a direction away from the elastic piece body 144. The spreading stroke of the supporting end part 145 is limited to avoid the connecting point between the pressing end part 143 and the elastic piece body 144 directly by contacting the base plate 15. So, the service life of the elastic piece 144 can be extended. When stop pressing the pressing end part 143, the supporting end part 145 abuts against one end of the base plate 15 and moves on the second surface 152 in a direction closing to the elastic piece body 144, which drives the elastic piece 14 back to the initial position. Thus, the elastic piece 14 and the button 16 would perform automatically returning back to the position before the pressing end part 143 is pressed.

In this embodiment, one end of each supporting end 145 away from the elastic piece body 144 abuts against the second surface 152 of the base plate 15. In this embodiment, each supporting end part 145 comprises a first supporting piece 1451 and a second supporting piece 1452. The first supporting piece 1451 is connected to the second side 144b of the elastic piece body 144 and extends in a direction away from the base plate 15. The second supporting piece 1452 is connected to one side of the first supporting piece 1451 away from the elastic piece body 144 and extends toward the base plate 15. One side of the second supporting piece 1452 away from the first supporting piece 1451 abuts against the second surface 152 of the base plate 15. The first supporting piece 1451 and the second supporting piece 1452 are both integrally formed. An angle is defined between the first supporting piece 1451 and the second supporting piece 1452. The spreading stroke of the first supporting piece 1451 and the second supporting piece 1452 is limited by the angle.

In one embodiment, one side of the second supporting piece 1452 away from the first supporting piece 1451 comprises a second bending part 1452a. The second bending part 1452a abuts on the second surface 152 of the base plate 15. The surface of the second bending part 1452a facing the base plate 15 is arc-shaped. In this way, not only the contact area between the second bending part 1452a and the base plate 15 can be increased but the wearing on the second surface 152 of the base plate 15 as the second supporting piece 1452 slides on the second surface 152 of the base plate 15 can also be avoided.

In one embodiment, the second surface 152 of the base plate 15 comprises a wear-resistant part 1521. The second bending part 1452a of each second supporting piece 1452 abuts against the second surface 152 of the base plate 15, preventing the second surface 152 of the base plate 15 from wearing as the second bending part 1452a moves on the second surface 152 of the base plate 15. The number of wear-resistant part 1521 in this embodiment is two. The two wear-resistant parts 1521 are disposed on the second surface 152 of the base plate 15 at intervals. Each second bending part 1452a abuts against the corresponding wear-resistant part 1521. The wear-resistant part 1521 provides a wear-resistant material on the second surface 152 of the base plate 15.

The configuration of the elastic piece 14 of this embodiment allows only a slight press on the button 16 to separate the connecting end part 142 of the elastic piece 14 from the elastic piece connecting part 111. So, the pressing stroke can be reduced and thereby reducing the overall size of the elastic piece 14. The pressed button 16 can be automatically returned to the initial position before pressing by the automatically returning elastic piece 14. Thus, extra restoring mechanism is no longer needed, the overall size and cost can be reduced effectively.

Back to FIG. 2 and FIG. 3, the housing 13 of this embodiment further comprises at least one cushioning elastic piece 133. The at least one cushioning elastic piece 133 is disposed on one side of the first opening 132 and extends into the first opening 132. In this embodiment, the number of cushioning elastic pieces 133 is two. One end of each cushioning elastic piece 133 is disposed at one side of the first opening 132 close to the test strip insertion hole 101 and extends in a direction away from the test strip insertion hole 101. In other words, the cushioning elastic piece 133 is disposed on the moving path of the sliding block 11. The extending direction of the cushioning elastic piece 133 is parallel to the moving path of the sliding block 11. When the connecting end part 142 is connected to the elastic piece connecting part 111, the cushioning elastic piece 133 abuts against the sliding block 11. When the connecting end part 142 is separated from the elastic piece connecting part 111, the elastic force of the elastomer 12 drives the sliding block 11 to move toward the test strip insertion hole 101. The cushioning elastic piece 133 first slows down the initial moving speed of the sliding block 11, and push the test strip 2 toward the outside of the test strip connector 10 at a low speed. In this way, rapid movement of the sliding block 11 can be avoided to reduce the wearing of the test strip connector 10 or the test strip 2 being ejected from the test strip connector 10.

In one embodiment, one end of the cushioning elastic piece 133 abutting against the sliding block 11 comprises a cushioning bump 1331. The cushioning bump 1331 abuts against the sliding block 11 to reduce the contact area between the cushioning elastic piece 133 and the sliding block 11, avoiding the cushioning elastic piece 133 hindering the movement of the sliding block 11.

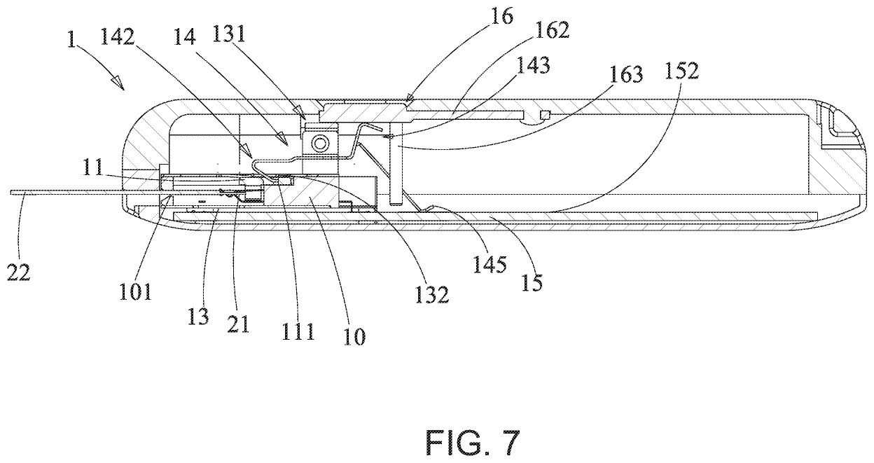

FIG. 7 is a cross-sectional view of a detecting device of the second embodiment of the present disclosure. As shown in the figure, the detecting device 1 of this embodiment differs from the detecting device of the first embodiment in the position of the base plate 15. The base plate 15 of this embodiment is not disposed between the housing 13 and the elastic piece 14. The base plate 15 is disposed below the housing 13 and is opposite to the elastic piece 14. The housing 13 provided with the test strip connector 10 is disposed on the second surface 152. The first connecting part 131 extends in a direction away from the second surface 152. The second connecting part 141 of the elastic piece 14 is rotatably connected to the first connecting part 131. The connecting end part 142 of the elastic piece 14 directly passes through the first opening 132 of the housing 13 and is movably connected to the elastic piece connecting part 111. The two supporting end parts 145 of the elastic piece 14 still extend toward the base plate 15 and abut against the second surface 152 of the base plate 15.

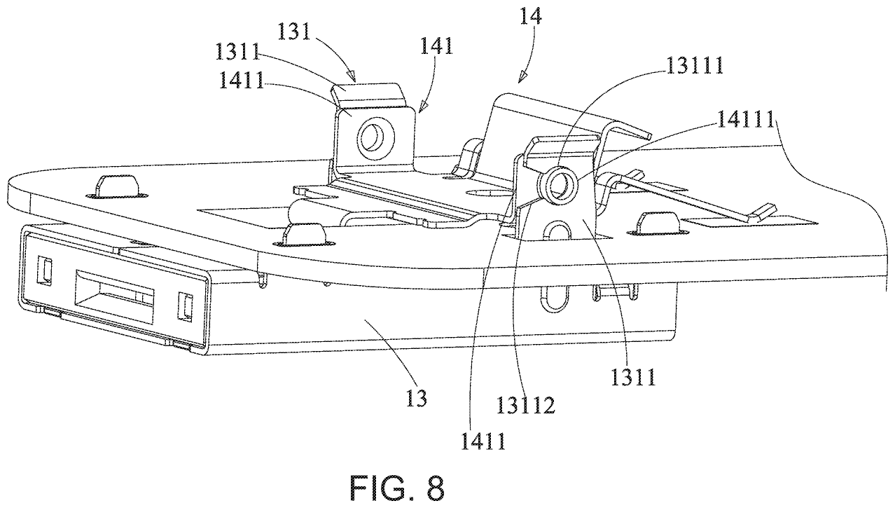

FIG. 8 is a combined perspective view of an elastic piece, a base plate, a housing, and a test strip connector of the third embodiment of the present disclosure. As shown in the figure, the detecting device 1 of this embodiment differs from the detecting device of the first embodiment in the configuration of the first connecting part 131 and the second connecting part 141. The first connecting part 131 of this embodiment also comprises two first connecting arms 1311. Each of the first connecting arms 1311 comprises a connecting hole 13111 and a notch 13112. The notch 13112 communicates with the connecting hole 13111 and penetrates one side of the first connecting arm 1311. The second connecting part 141 also comprises two second connecting arms 1411. Each of the second connecting arms 1411 comprises a connecting shaft 14111. When the second connecting arm 1411 is disposed on the corresponding first connecting arm 1311, the connecting shaft 14111 of the second connecting arm 1411 is disposed in the connecting hole 13111 of the corresponding first connecting arm 1311 through the notch 13112. That is, the connecting shaft 14111 enters the connecting hole 13111 through the notch 13112. The second connecting part 141 of the elastic piece 14 of this embodiment is assembled to the first connecting part 131 of the housing 13 along a direction parallel to the second surface 152 of the base plate 15. In one embodiment, the width of one end of the notch 13112 connected to the connecting hole 13111 is narrower than the diameter of the connecting hole 13111 and also narrower than the outer diameter of the connecting shaft 14111. In this way, the detaching of the connecting shaft 14111 from the connecting hole 13111 can be avoided.

In one embodiment, the width of one end of the notch 13112 away from the connecting hole 13111 is greater than the width of one end of the notch 13112 connected to the connecting hole 13111. So, the width of the notch 13112 gradually increases outward starting from the connecting hole 13111. The width of one end of the notch 13112 away from the connecting hole 13111 is greater than or equal to the outer diameter of the connecting shaft 14111. So, the connecting shaft 14111 could easily enter the notch 13112. The configuration of the first connecting part 131 and the second connecting part 141 of this embodiment can be applied to the first connecting part and the second connecting part of the second embodiment.

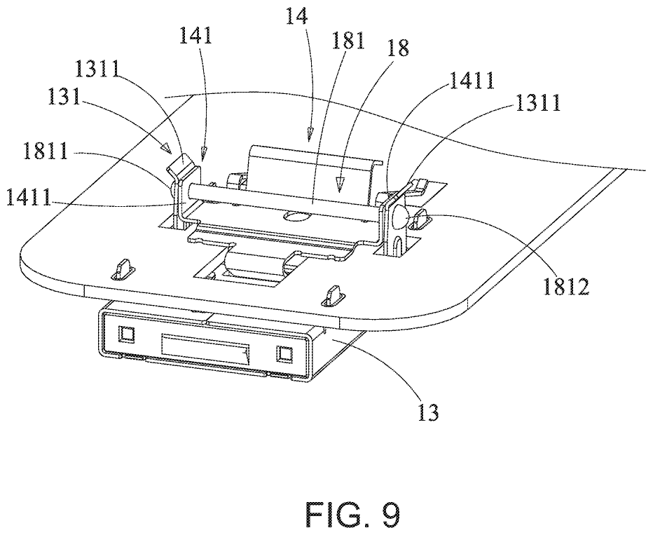

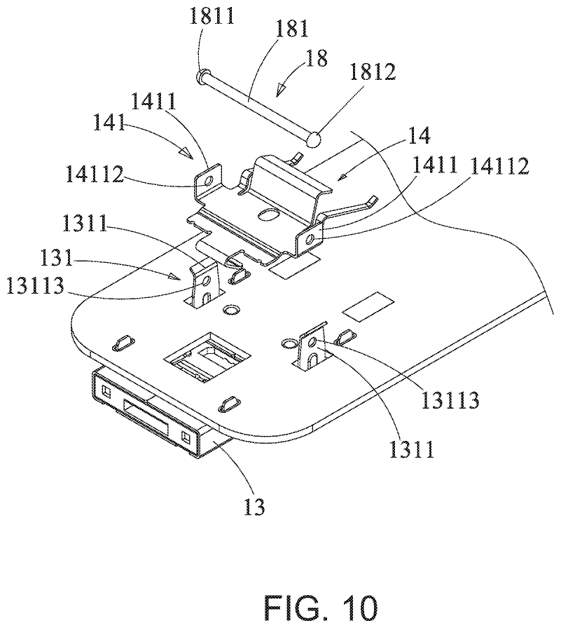

FIG. 9 and FIG. 10 are a combined perspective view and a partial exploded view of an elastic piece, a base plate, a housing, and a test strip connector of the fourth embodiment of the present disclosure. As shown in the figure, the detecting device of this embodiment differs from the detecting device of the first embodiment in the configuration of the first connecting part 131 and the second connecting part 141. The first connecting part 131 of this embodiment also comprises two first connecting arms 1311. Each of the first connecting arms 1311 comprises a first connecting hole 13113. The second connecting part 141 also comprises two second connecting arms 1411. Each of the second connecting arms 1411 of this embodiment comprises a second connecting hole 14112. When the first connecting part 131 is connected to the second connecting part 141, the two second connecting arms 1411 are disposed between the two first connecting arms 1311, and each of the second connecting arms 1411 is adjacent to the corresponding first connecting arm 1311, forming two sets of adjacent first connecting arm 1311 and second connecting arm 1411. The first connecting hole 13113 of each of the first connecting arms 1311 corresponds to the second connecting hole 14112 of the corresponding second connecting arm 1411. Finally, the securing component 18 passes through each of the first connecting holes 13113 of the first connecting part 131 and each of the second connecting holes 14112 of the second connecting part 141. That is, the securing component 18 passes through the first connecting hole 13113 of each of the adjacent first connecting arms 1311 and the second connecting hole 14112 of each of the adjacent second connecting arms 1411, allowing the first connecting part 131 to be rotatably connected to the second connecting part 141.

In one embodiment, the securing component 18 of this embodiment comprises a securing shaft 181. Two ends of the securing shaft 181 respectively comprise a first stopper 1811 and a second stopper 1812. When the securing shaft 181 passes through and is disposed in the first connecting hole 13113 of each of the adjacent first connecting arms 1311 and the second connecting hole 14112 of each of the adjacent second connecting arms 1411, the first stopper 1811 abuts against an outer surface of the first connecting arm 1311, and the second stopper 1812 abuts against an outer surface of the other first connecting arm 1311. In this embodiment, the first stopper 1811 is formed on one end of the securing shaft 181 before the securing shaft 181 passes through the first connecting part 131 and the second connecting part 141. After the securing shaft 181 passes through the first connecting part 131 and the second connecting part 141, the outer diameter or width of the first stopper 1811 is greater than the diameter of the first connecting hole 13113. The first stopper 1811 abuts against an outer surface of the corresponding first connecting arm 1311 to position the securing shaft 181. The second stopper 1812 is formed on the other end of the securing shaft 181 by hot riveting after the securing shaft 181 passes through and is disposed in the first connecting part 131 and the second connecting part 141. The outer diameter or width of the second stopper 1812 is greater than the diameter of the first connecting hole 13113. The second stopper 1812 abuts against an outer surface of the corresponding first connecting arm 1311 to secure the securing shaft 181 to the first connecting part 131 and the second connecting part 141. In this embodiment, the first stopper 1811 and the second stopper 1812 are both bumps. The configuration of the first connecting part 131, the second connecting part 141, and the securing component 18 of this embodiment can be applied to the first connecting part and the second connecting part of the second embodiment.

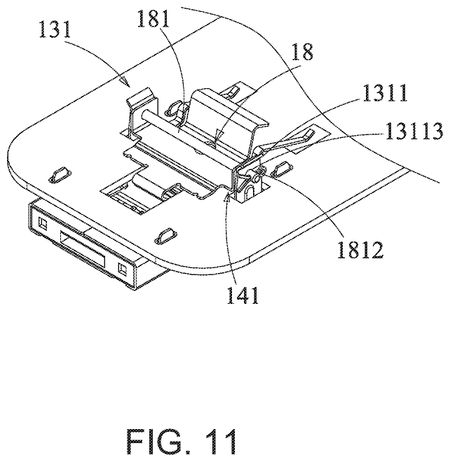

FIG. 11 is a combined perspective view of an elastic piece, a base plate, a housing, and a test strip connector of the fifth embodiment of the present disclosure. As shown in the figure, the detecting device of this embodiment differs from the detection device of the fourth embodiment in the second stopper 1812, which is an insertion pin. The second stopper 1812 can be separated from the securing shaft 181. So, the securing shaft 181 can be secured to the first connecting part 131 and the second connecting part 141, which is convenient for installation and disassembly. The width of the second stopper 1812 of this embodiment is greater than the diameter of the first connecting hole 13113. Thus, the second stopper 1812 can abut against a surface of the first connecting arm 1311, which allows the securing shaft 181 to be secured to the first connecting part 131 and the second connecting part 141. The first stopper 1811 can also be an insertion pin. Or, the first stopper 1811 is an insertion pin, meanwhile, the second stopper 1812 is a bump. The configuration of the first connecting part 131, the second connecting part 141, and the securing component 18 of this embodiment can be applied to the first connecting part and the second connecting part of the second embodiment.

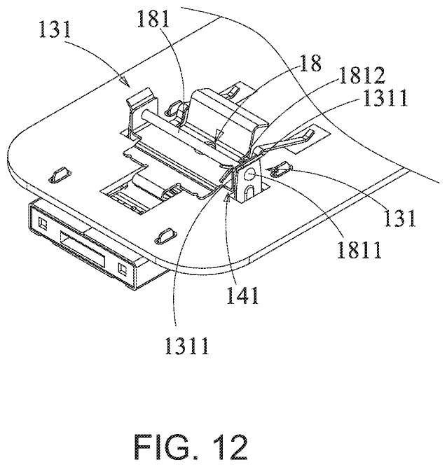

FIG. 12 is a combined perspective view of an elastic piece, a base plate, a housing, and a test strip connector of the sixth embodiment of the present disclosure. As shown in the figure, the detecting device of this embodiment differs from the detection device of the fifth embodiment in the position of the first stopper 1811 and the second stopper 1812. In this embodiment, the first stopper 1811 abuts against an outer surface of the first connecting part 131, and the second stopper 1812 abuts against an inner surface of the second connecting part 141. The first stopper 1811 abuts against an outer surface of one of the two first connecting arms 1311. The second stopper 1812 abuts against an inner surface of one of the two second connecting arms 1411. The second connecting arm 1411 abutted by the second stopper 1812 is adjacent to the first connecting arm 1311 abutted by the first stopper 1811. In this embodiment, the first stopper 1811 is a bump, and the second stopper 1812 is an insertion pin. The second stopper 1812 can be disposed separately from the securing shaft 181 to facilitate the assembly of the securing shaft 181.

In summary, the present disclosure provides a detecting device. By disposing a sliding block and an elastomer on a test strip connector and by controlling the position of the sliding block by whether a connecting end part of an elastic piece is connected to an elastic connecting part of the sliding block or not, a detecting device can detect the sample on the test strip when the connecting end part of the elastic piece is connected to the elastic connecting part of the sliding block. When the connecting end part of the elastic piece is detached from the elastic connecting part of the sliding block, the sliding block can be pushed by the elastomer to allow automatically returning of the test strip.

It is to be understood that the term “comprises”, “comprising”, or any other variants thereof, is intended to encompass a non-exclusive inclusion, such that a process, method, article, or device of a series of elements not only comprise those elements but also comprises other elements that are not explicitly listed, or elements that are inherent to such a process, method, article, or device. An element defined by the phrase “comprising a . . . ” does not exclude the presence of the same element in the process, method, article, or device that comprises the element.

Although the present disclosure has been explained in relation to its preferred embodiment, it does not intend to limit the present disclosure. It will be apparent to those skilled in the art having regard to this present disclosure that other modifications of the exemplary embodiments beyond those embodiments specifically described here may be made without departing from the spirit of the disclosure. Accordingly, such modifications are considered within the scope of the disclosure as limited solely by the appended claims.

What is claimed is:

1. A detecting device, comprising:
a test strip connector;
a sliding block slidably disposed on the test strip connector comprising an elastic piece connecting part;
an elastomer disposed between the sliding block and the test strip connector, the extending direction of the elastomer being parallel to the sliding direction of the sliding block;
a housing disposed on the test strip connector, comprising a first connecting part and a first opening, the elastic piece connecting part of the sliding block being exposed from the first opening; and
an elastic piece comprising a second connecting part, a connecting end part and a pressing end part, the connecting end part and the pressing end part being disposed on two sides of the second connecting part rotatably connected to the first connecting part, the connecting end part being movably connected to the elastic piece connecting part;
wherein an end part of a test strip is inserted into the test strip connector; the connecting end part is separated from the elastic piece connecting part after the pressing end part is pressed; the elastomer pushes the sliding block which drives an end part of the test strip out of the test strip connector.

2. The detecting device according to claim 1, wherein the test strip connector comprises a test strip insertion hole and a test strip accommodating space communicating with the test strip insertion hole; the end part of the test strip is inserted into the test strip accommodating space through the test strip insertion hole; the sliding block is disposed on one side of the test strip accommodating space; the sliding block corresponds to the end part of the test strip.

3. The detecting device according to claim 2, wherein the sliding block comprises a pushing part disposed in the test strip accommodating space to correspond to the end part of the test strip.

4. The detecting device according to claim 2, wherein the test strip connector comprises two sliding grooves respectively disposed on two sides of the test strip accommodating space; two sides of the sliding block are respectively disposed in the two sliding grooves; the elastomer is disposed on the sliding groove; two ends of the elastomer respectively abut against the sliding block and a sidewall of the sliding groove.

5. The detecting device according to claim 2 further comprises a base plate comprising a first surface, a second surface, and a second opening; the first surface is opposite to the second surface; the second opening passes through the first surface and the second surface; the test strip connector is disposed on the first surface; the first connecting part of the housing passes through the base plate; the first connecting part protrudes from the second surface; the second opening corresponds to the first opening; the elastic piece connecting part is exposed from the second opening; the second connecting part of the elastic piece is rotatably connected to the first connecting part; the elastic piece is disposed on the second surface; the connecting end part of the elastic piece is movably connected to the elastic piece connecting part.

6. The detecting device according to claim 2 further comprises a base plate comprising a first surface and a second surface corresponding to the first surface; the test strip connector is disposed on the second surface; the second connecting part of the elastic piece is rotatably connected to the first connecting part; the connecting end part of the elastic piece is movably connected to the elastic piece connecting part.

7. The detecting device according to claim 5, wherein the base plate is a circuit board; the test strip connector comprises a plurality of terminals; one end of the terminals is electrically connected to the base plate while the other end is disposed in the test strip accommodating space.

8. The detecting device according to claim 6, wherein the base plate is a circuit board; the test strip connector comprises a plurality of terminals; one end of the terminals is electrically connected to the base plate while the other end is disposed in the test strip accommodating space.

9. The detecting device according to claim 1 further comprises a button disposed on one side of the elastic piece away from the connecting end part; the button is also disposed on one side of the pressing end part.

10. The detecting device according to claim 9 further comprises an outer housing accommodating the test strip connector, the sliding block, the elastomer, the housing, the base plate, and the elastic piece; the outer housing comprises a button opening; the button is disposed in the button opening.

11. The detecting device according to claim 10, wherein the button comprises a button body and a securing piece connected to one side of the button body; the button body is disposed in the button opening; the securing piece is secured to an inner surface of the outer housing.

12. The detecting device according to claim 11, wherein the button further comprises at least one limiting cylinder disposed on a surface of the button body facing the base plate.

13. The detecting device according to claim 2, wherein the elastic piece further comprises an elastic piece body; the connecting end part comprises a first connecting piece and a second connecting piece; the first connecting piece is connected to the elastic piece body; the second connecting piece is connected to one side of the first connecting piece away from the elastic piece body and extends in a direction of the test strip accommodating space; one side of the second connecting piece away from the first connecting piece is movably connected to the elastic piece connecting part.

14. The detecting device according to claim 13, wherein when the connecting end part is connected to the elastic piece connecting part, an angle is defined between the second connecting piece and the first connecting piece; the angle is greater than 45 degrees and less than 90 degrees.

15. The detecting device according to claim 13, wherein the connecting end part further comprises a third connecting piece connected to one side of the second connecting piece away from the first connecting piece; when the connecting end part is connected to the elastic piece connecting part, the third connecting piece is parallel to the first connecting piece to abut against a sidewall of the elastic piece connecting part.

16. The detecting device according to claim 9, wherein the pressing end part comprises a pressing elastic piece connected to the elastic piece body; the pressing elastic piece extends towards the button; one side of the pressing elastic piece away from the elastic piece body is disposed on one side of the button.

17. The detecting device according to claim 16, wherein the side of the pressing elastic piece away from the elastic piece body comprises a first bending part disposed on one side of the button.

18. The detecting device according to claim 16, wherein the elastic piece further comprises two supporting end parts on the same side as the pressing end part; the two supporting end parts are disposed on two sides of the pressing end part; the two supporting end parts abut against the second surface of the base plate.

19. The detecting device according to claim 18, wherein each of the supporting end parts comprises a first supporting piece and a second supporting piece; the first supporting piece is connected to the elastic piece body; the first supporting piece extends in a direction departing from the base plate; the second supporting piece is connected to one side of the first supporting piece away from the elastic body; the second supporting piece extends in a direction closing to the base plate; one side of the second supporting piece away from the first supporting piece abuts against the second surface of the base plate.

20. The detecting device according to claim 19, wherein one side of the second supporting piece is provided with a second bending part abutting against the second surface of the base plate.

21. The detecting device according to claim 20, wherein the second surface of the base plate comprises a wear-resistant part; the two second bending parts abut against the wear-resistant part.

22. The detecting device according to claim 2, wherein the housing further comprises at least one cushioning elastic piece disposed on one side of the first opening; the at least one cushioning elastic piece extends into the first opening; when the connecting end part is connected to the elastic piece connecting part, the cushioning elastic piece abuts against the sliding block.

23. The detecting device according to claim 1, wherein the first connecting part comprises two first connecting arms oppositely disposed; each of the first connecting arms comprises a connecting hole; the second connecting part comprises two second connecting arms oppositely disposed; each of the second connecting arms comprises a connecting shaft; the connecting shaft of each of the second connecting arms is disposed in the connecting hole of the corresponding first connecting arm.

24. The detecting device according to claim 23, wherein each of the first connecting arms comprises a notch communicating with the connecting hole; the connecting shaft of each of the second connecting arms is disposed in the connecting hole of the corresponding first connecting arm through the notch.

25. The detecting device according to claim 1, wherein the first connecting part comprises two first connecting arms oppositely disposed; each of the first connecting arms comprises a first connecting hole; the second connecting part comprises two second connecting arms oppositely disposed; each of the second connecting arms comprises a second connecting hole; the first connecting hole of each of the first connecting arms corresponds to the second connecting hole of the corresponding second connecting arm. A securing component passes through the first connecting hole of the first connecting part and the second connecting hole of the second connecting part.

26. The detecting device according to claim 25, wherein the securing component comprises a securing shaft; two ends of the securing shaft comprises a first stopper and a second stopper; the securing shaft penetrates and is disposed in the first connecting hole and the second connecting hole; the first stopper abuts against an outer surface of the first connecting part; the second stopper abuts against an inner surface of the second connecting part.

27. The detecting device according to claim 25, wherein the securing component comprises a securing shaft; two ends of the securing shaft comprises a first stopper and a second stopper; the securing shaft penetrates and is disposed in the first connecting hole and the second connecting hole; the first stopper abuts against an outer surface of the first connecting arm; the second stopper abuts against an outer surface of the second connecting arm.

28. The detecting device according to claim 26, wherein the first stopper and the second stopper are an insertion pin or a bump.

29. The detecting device according to claim 27, wherein the first stopper and the second stopper are an insertion pin or a bump.

30. The detecting device according to claim 1, wherein the elastomer is a spring.
